(19) United States
(12) Patent Application Publication
(10) Pub. No.: US 2024/0096481 A1
(43) Pub. Date: Mar. 21, 2024

(54) SCHEDULING HEALTHCARE-RELATED SERVICES, EMR ACCESS, AND WOUND DETECTION

(71) Applicant: POSTOP CARE LLC, Las Vegas, NV (US)

(72) Inventor: Adam Nadelson, Watermill, NY (US)

(73) Assignee: POSTOP CARE LLC, Las Vegas, NV (US)

(21) Appl. No.: 18/371,410

(22) Filed: Sep. 21, 2023

Related U.S. Application Data
(60) Provisional application No. 63/376,587, filed on Sep. 21, 2022.

(51) Int. Cl. G16H 40/20; G06T 7/00; G16H 10/20; G16H 10/60; G16H 40/67

(52) U.S. Cl. CPC G16H 40/20; G06T 7/0016; G16H 10/20; G16H 10/60; G16H 40/67; G06T 2207/20081

(57) ABSTRACT

A system and method for providing healthcare-related services. The method includes receiving identifying data associated with a user. The method further requesting information from a patient, which may be in the form of questions. The method also includes scheduling a telemedicine appointment with a patient, which may be before or after a surgery. The method further includes receiving images of a patient, which may include a wound caused by the surgery. The method additionally includes a provider receiving the answers to the questions and images of the patient. The method also includes determining whether the wound is abnormal.

### BACKGROUND

The healthcare industry is undergoing a meaningful transformation built upon an underlying digital foundation. This digital foundation paves the way for a digital healthcare journey for patients, clinicians, and healthcare organizations. Typically, many patients prefer to minimize or completely avoid trips to a healthcare provider (e.g., hospital, clinic, physician, nurse, etc.) before or after a medical procedure. Especially, when feeling unwell, distressed, exhausted, etc. Some reasons include preferring to avoid unnecessary travel, long wait times, anxiety, and unnecessary exposure to various ailments and infections. Even worse, is to make such a trip frequently and/or multiple times.

Further, from time to time, or even frequently, a patient's feedback is not accurately captured into their chart (medical record) by the healthcare providers. Because feedback can be at least somewhat misunderstood or misconstrued, and/or sometimes partially or completely omitted.

Next, traditional methods of predicting wound healing have been limited. Healthcare providers can only predict the course of healing with only so much accuracy/certainty. This problem is compounded by the fact that the wound(s) are checked only so often, in part because of the busy schedules of the healthcare providers and the prohibitive nature of frequent patient visits.

### SUMMARY

A need exists for a digital platform that can connect patients with providers that can provide pre-, intra-, and post-op telemedicine care.

Today's patients can receive meaningful care from the comfort of their own home—particularly as the quality of video conferencing hardware and software has improved so dramatically in recent years. With better technology, patients can receive care from the comfort of their own home. When deployed correctly, this allows providers to continuously monitor a patient and their wounds without the need for the patient to come into the medical office as frequently or total times as they traditionally would after a procedure is performed to ensure proper healing.

Moreover, systems today provide more autonomy to patients. For example, rather than spend a costly amount of time with a medical assistant that asks them routine questions, such as whether they have a fever, today patients can easily take their temperature at home and enter the results through an online form (or through an Internet-connected thermometer itself). In other words, as technology improves, the need for a trip for care can be reduced.

In some embodiments, a system may receive patient-identifying information and payment information. Payment information may include a card payment, and/or an approval by an insurance carrier. The system may also schedule telemedicine appointments, which may be automatically scheduled before and/or after a medical procedure, and the time/date of those appointments may be based on the procedure or the provider's preference.

The system may send questions to a patient before a telemedicine visit. These questions may be about a patient's current health. The patient may then send responses to the questions, which may include written answers and/or images such as a surgical wound.

The system may connect a patient to a provider remotely (e.g., using telemedicine). The provider may review the patient's answers to the provided questions and/or images, and the provider may request additional images and/or information from the patient.

In various embodiments, the systems and methods described herein may identify infections or other abnormalities associated with the patient's responses to questions and/or images. A system may include a machine learning module that is able to identify infections and abnormalities, and notify a provider when it has identified those infections or abnormalities.

Other embodiments will be apparent from the following description and the appended claims.

### BRIEF DESCRIPTION OF THE DRAWINGS

Embodiments are illustrated by way of example, and not by way of limitation, in the figures of the accompanying drawings and in which like reference numerals refer to similar elements.

FIG. 1 shows a schematic diagram of a system, in accordance with one or more embodiments.

FIG. 2 shows an exemplary screenshot of a user interface including a QR code to help the patient access their electronic medical record.

FIG. 3 shows an exemplary screenshot of a user interface including questions for a patient provided by a healthcare platform.

FIG. 4 shows an exemplary screenshot of a user interface presented to a patient including dates that a provider would like to meet with the patient.

FIG. 5 shows an exemplary image taken by a patient of a body part.

FIG. 6 shows a flowchart of a method for scheduling an appointment.

FIG. 7 shows a flowchart for initiating a telemedicine appointment.

FIGS. 8 and 9 show a computing system and network architecture in accordance with one or more embodiments.

### DETAILED DESCRIPTION

A portion of the disclosure of this patent document may contain material that is subject to copyright protection. The copyright owner has no objection to the facsimile reproduction by anyone of the patent document or the patent disclosure, as it may appear in the Patent and Trademark Office patent file or records, but otherwise reserves all copyrights whatsoever.

Specific embodiments will now be described in detail with reference to the accompanying figures. Like elements in the various figures are denoted by like reference numerals for consistency. In the following detailed description of embodiments, numerous specific details are set forth in order to provide a more thorough understanding of the invention. While described in conjunction with these embodiments, it will be understood that they are not intended to limit the disclosure to these embodiments. On the contrary, the disclosure is intended to cover alternatives, modifications and equivalents, which may be included within the spirit and scope of the disclosure as defined by the appended claims. It will be apparent to one of ordinary skill in the art that the invention can be practiced without these specific details. In other instances, well-known features have not been described in detail to avoid unnecessarily complicating the description.

It should be appreciated that while the present disclosure may individually refer to preoperative, intraoperative, or postoperative care, embodiments herein may apply to or involve some or all of preoperative, intraoperative, or postoperative care. For example, if a concept is discussed with reference to postoperative care, then it may also apply to preoperative care. In one example, if charting based on a QR code is discussed only in a postoperative care context, the QR code charting may also apply to preoperative care even if not explicitly mentioned.

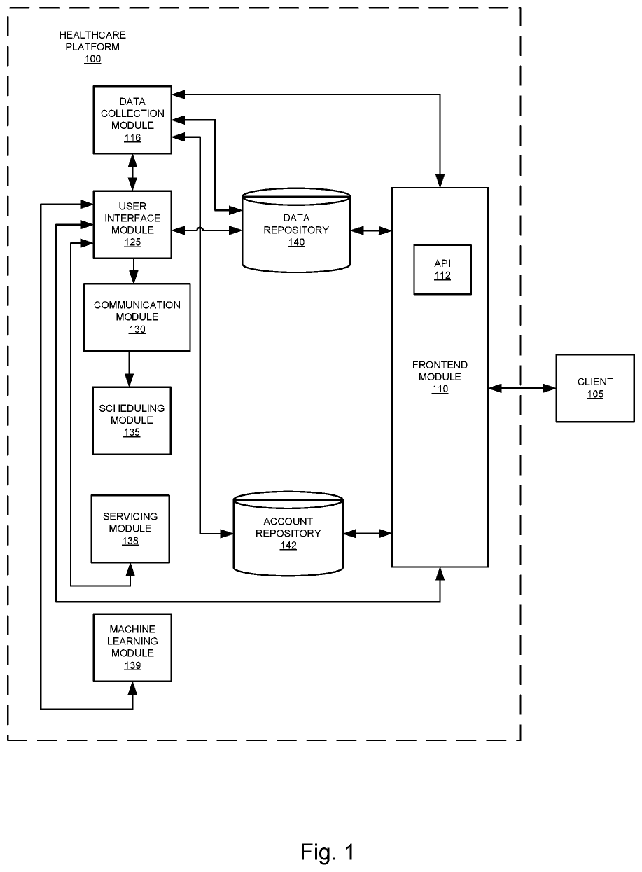

FIG. 1 shows a healthcare platform 100 and a client computing device 105 in accordance with one or more embodiments. As shown in FIG. 1, the healthcare platform 100 has multiple components including a frontend module 110 with an application programming interface (API) 112, a data collection module 116, a user interface module 125, a communication module 130 (operable to be HIPAA-compliant/satisfy HIPAA communication requirements), a scheduling module 135, servicing module 138, ranking module 139, a data repository 140, and an account repository 142.

In one or more embodiments, the healthcare platform 100 is a platform for communication between a patient and a provider. The healthcare platform 100 may be configured to enable a patient to communicate in “real-time” with a provider, e.g., to converse with them with a minimal delay. In other words, the healthcare platform 100 may allow a user to submit messages and may display the messages to one or more other users (e.g., clinicians, admin staff, physicians, or other providers) within a reasonable time frame so as to facilitate a live conversation between the patient and provider.

For example, after a patient has a procedure performed such as but not limited to blepharoplasty, rhinoplasty, or breast augmentation, platform 100 can facilitate communication between a provider and a patient (e.g., via communication module 130). Not only can platform 100 save time for a patient by eliminating the need for travel, but platform 100 can also save time and costs for a provider because a patient can provide an accurate account of their symptoms without the need for a physician's assistant to ask questions. Moreover, platform 100 can provide high-quality images of a patient's wounds such that a provider can feel confident the patient is healing appropriately.

The healthcare platform 100 may include Electronic Medical Record (EMR) data, Electronic Health Record (EHR) data, History of Present Illness (HPI) data (collectively referred to as “EMR” data herein), Examination Assessment data, and/or Diagnosis & Plan data. In some embodiments, the healthcare platform 100 is an EMR/HER/HPI system and/or Examination Assessment and/or Diagnosis & Plan system. The healthcare platform 100 may include EMR data for patients corresponding to different geographies (e.g., ZIP codes, cities, counties, states, regions, etc.).

Healthcare platform 100 may be configured to receive an approval from an insurance carrier or otherwise receive a payment. In some embodiments, a code (e.g., a QR/“quick response” code) may be generated that provides a patient access to their EMR data, or at least the ability to add to it. It should be appreciated that other types of codes or linking pointers are possible, for example, bar codes, serial numbers, unique images, NFC (near field communication) tags, SnapTags, Bluetooth beacons, etc. This code may be generated in response to receiving an approval from an insurance carrier or a payment. In one or more embodiments, the code is unique/is a unique identifier. For example, there are no and/or will not be any other equivalent codes generated. The code may include a unique Internet hyperlink (or a deep link that sends the patient directly to a smartphone/desktop app instead of a website) that provides access to the patient's EMR data. Access to their EMR data may include read and/or write permissions (it should be appreciated that “their EMR data” can mean “the patient's EMR data” herein). For example, the patient may submit information about their symptoms or their recovery which are added to their medical chart.

Further, a patient may provide data to healthcare platform 100 such as answers to questions, which may relate to preoperative, intraoperative, and/or postoperative care (or a specific medical procedure). It is contemplated that a patient may also provide information captured by a device such as an electrocardiogram machine. Such data may be provided to a patient's EMR via communication module 130 and/or data collection module 116.

The healthcare platform 100 may provide a patient with directions, including whether the patient has completed all of the questions provided to them. Elsewhere, an administrator may use healthcare platform 100 to verify that the data was received correctly, spot any issues, and/or approve any laboratory work. In some embodiments, once an administrator reviews and/or approves of the data provided by the patient, the data may be sent to a physician or other provider who may also review questions, and perhaps additional data such as images captured by the patient that show their wounds so the provider may look for any signs of infection.

In some embodiments, the images captured by the patient may be of wounds created during a medical procedure (e.g., a surgical wound) or otherwise (e.g., an accident, infection, and/or blunt force). The images may be used by a provider to determine whether there is an infection, problem with the healing (e.g., a wound's healing progress is less than desired), or other abnormality. The patient may answer questions about the wound (e.g., whether and how it is painful, whether the wound area's temperature is elevated, etc.) or otherwise submit information about the wound, and the patient information submission(s) will be added to their EMR.

In some embodiments described herein, the healthcare platform 100 may determine whether a wound includes an infection or other abnormality based on an algorithm that employs machine learning and/or artificial intelligence. For example, a comparison of an image of a wound with other images of other wounds using a machine learning algorithm. In other words, an algorithm may be implemented that receives images of multiple wounds, and by using machine learning, the algorithm is able to recognize certain characteristics of wounds such as the type of wounds they are, the severity of the wounds, the stage of healing of the wound, etc. The machine learning may benefit from metadata about the wounds to aid in training of the machine learning. Healthcare platform 100 can then receive an image of a wound from a user's device, and then apply the algorithm to the image to determine characteristics of the wound in the image such as what type of wound it is, or the severity of the wound.

In some embodiments, the hardware and/or software used by a patient and/or provider must be of a certain quality. And/or, in some examples, the healthcare platform 100 may be able to determine whether an image provided by a patient is of a threshold quality. In some embodiments, an object of known color(s) and/or pattern(s), such as a white card for calibrating white balance, may be included in an image (which may also include a wound) in order to calibrate an image to increase its fidelity. If an image is not above a threshold quality, the healthcare platform 100 may notify a patient that the image is of insufficient quality and/or must be recaptured. Whether the initial image submission or a recapture, the healthcare platform 100 may provide an example photo to help guide the patient in how to set up and capture the image.

In some embodiments, the healthcare platform 100 (and/or AI/algorithm) uses the metadata of the image file in its analysis. For example, the EXIF (Exchangeable Image File Format) data of an image file may include information such as camera settings (aperture setting, exposure setting, ISO setting, lens, depth of field, etc.), date/time the photo was taken, etc. In some embodiments the EXIF data of the image may be used to create a derivative of an image of a wound such that the healthcare platform can better identify characteristics of the wound. For example, the healthcare platform 100 may adjust the brightness or contrast of an image based on the image's EXIF information in order to identify characteristics of a wound with greater accuracy.

The data collection module 116 may be configured to collect data about a user of the healthcare platform 100. The data may include healthcare-related information about the user. For example, the data collection module 116 may collect a user's vital information such as heart rate, temperature, blood pressure, blood oxygen level, etc. Such data may be stored in the data repository 140. Data collection module 116 may also receive and/or provide information to a patient's EMR. In some embodiments, the data collection module 116 may collect information about a user's medical history, age, race, familial medical history, etc. Further, the data collection module 116 may collect socio-economic information, financial information, and/or information about a location or neighborhood that a user lives in.

The user interface module 125 may be configured to render a user interface usable by a patient and/or provider. The user interface module 125 may be configured to facilitate the communication of messages of the healthcare platform 100 (e.g., between patients, providers, physicians, nurses, etc.). The user interface module 125 may be configured to be HIPAA-compliant and/or satisfy HIPAA communication requirements.

In addition or in place, as described above, the communication module 130 may be configured to facilitate the communication of messages of, to, and from the healthcare platform 100. For example, the communication module 130 may be configured to transmit and receive text messages (e.g., cellular phone SMS or MMS), short code messages, or internet-based notification (iOS app or mobile web notification) to and from client 105. The communication module 130 may be configured to be HIPAA-compliant (e.g., secured and/or password-protected links store patient health information within the healthcare platform 100). Both the user interface module 125 and the communication module 130 may communicate the same, similar, or different messages.

The scheduling module 135 may be configured to facilitate the scheduling of a healthcare related service requested by the user via the healthcare platform 100. For example, scheduling module 135 may be configured to create an appointment for a patient in response to the patient completing questions and/or sending images of their wounds to a provider. Scheduling module 135 may also be used by a provider to determine what times the provider may virtually meet with a patient (e.g., a telemedicine appointment). In some embodiments, a provider may enter instructions indicating that they'd like to meet with a patient on certain days or at a certain cadence (e.g., the first day after a procedure, the second day after a procedure, the fourth day after the procedure, the seventh day after a procedure, the fourteenth day after a procedure, and approximately the 31st day after a procedure). In such an embodiment, scheduling module 135 may determine times that the provider is available on those days, and request approval from a patient. Further, in some embodiments, the cadence of the appointments may be based upon the type of procedure performed. For instance, healthcare platform 100 may suggest/generate more appointments if the patient received a rhinoplasty as opposed to a liposuction procedure.

The servicing module 138 may be configured to receive a service request for a healthcare-related service from a user. The service request may be received, for example, via client 105. In some embodiments, the service request causes the healthcare platform 100 to request a payment or authorization from an insurance carrier.

The machine learning module 139 may be configured to receive images or data external to the healthcare platform, and in some embodiments optionally including patient and/or healthcare provider comments. Based on these images and data, machine learning module 139 may be able to identify infections or other abnormalities of images provided by a patient. For example, the machine learning module 139 may receive a variety of images that show infected and non-infected wounds, and gain the ability to determine/estimate whether an image of a wound provided by a patient is infected or determine/estimate other characteristics about the wound. The machine learning module 139 may be “trained” on such determinations/estimations by analyzing multiple images (and corresponding data) where the determinations/estimations are already known (e.g., from past human healthcare provider analysis), so as to “teach” the machine learning module 139.

In some embodiments, additional appointments may be scheduled based on the determination/estimation by the healthcare module. Further, in some embodiments, the machine learning module 139 may provide a provider with its determination (e.g., the healthcare platform may notify the provider that it suspects the wound is infected, and allows the provider to use that information however they wish).

Although the components of the healthcare platform 100 are depicted as being directly communicatively coupled to one another, this is not necessarily the case. For example, one or more of the components of the healthcare system 100 may be communicatively coupled via a distributed computing system, a cloud computing system, or a networked computer system communicating via the Internet.

Although only one healthcare platform 100 is illustrated, it should be appreciated that this one healthcare platform may represent many healthcare platforms, arranged in a central or distributed fashion. For example, such a healthcare platform may be organized as a central cloud and/or may be distributed geographically or logically to edges of a system such as a content delivery network or other arrangement. It is understood that virtually any number of intermediary networking devices, such as switches, routers, servers, etc., may be used to facilitate communication.

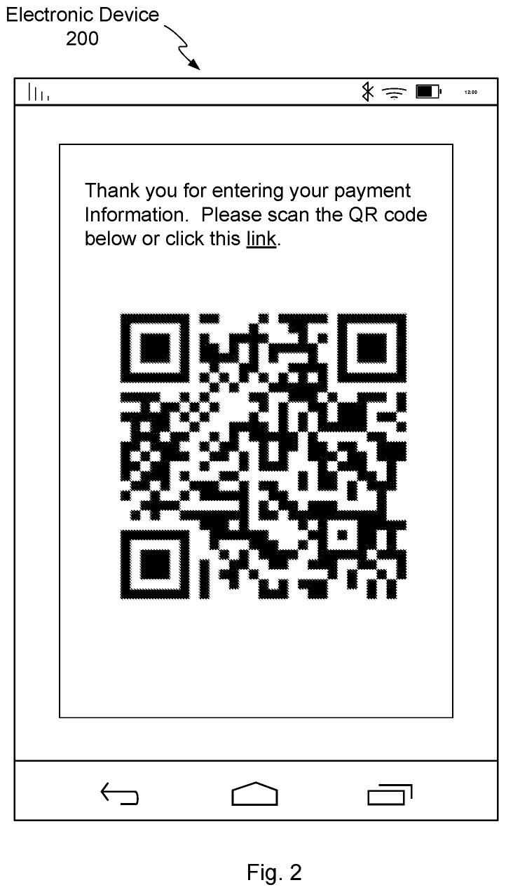

FIG. 2 shows an exemplary screenshot of an electronic device 200 including a user interface with a QR code and a link that allows a user to access their electronic medical record. For example, a user may provide healthcare platform 100 with payment information, insurance information (or an insurance carrier may provide healthcare platform 100 with an approval), and healthcare platform may provide a patient with a link to access their EMR, questions for them to answer, and/or requests for images (e.g., of a wound).

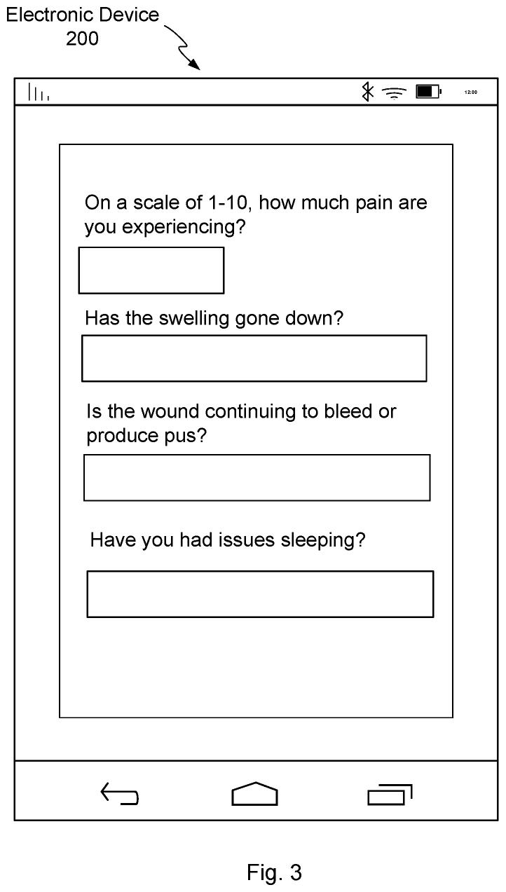

FIG. 3. shows an exemplary screenshot of an electronic device 200 including a user interface with a form including questions for a patient. The example shown in FIG. 3 may be generated by the user interface module 125 (FIG. 1). As described above, rather than talking directly to a provider, a patient may complete a questionnaire provided to them (e.g., the one shown in FIG. 3). The answers the patient provides to these questions may be inputted into their EMR, and may also be shown to their provider (e.g., a nurse, a physician, an insurance carrier). In some embodiments, a patient may not be able to have a telemedicine medical procedure follow-up appointment unless they answer the questions.

It should be understood that unlike many applications, in some embodiments described herein, patients are able to enter information that modifies their EMR directly. In other words, rather than let data get stale (e.g., if a provider copies and pastes, or a pharmacy simply takes the same information provided weeks previously), the instant disclosure describes a way for patients to enter information that modifies their EMR in real-time, preventing a loss in accuracy.

In some embodiments, the questions may prompt a user to capture images of themselves, which may or may not include a surgical wound. Images may be taken before or after a medical procedure. Further, a user may receive instructions on how to take images from healthcare platform 100. For example, if a patient is going to have a liposuction procedure performed, healthcare platform 100 may ask the patient to take one or more images of themselves, and the healthcare platform may ask the patient to take multiple images from multiple angles.

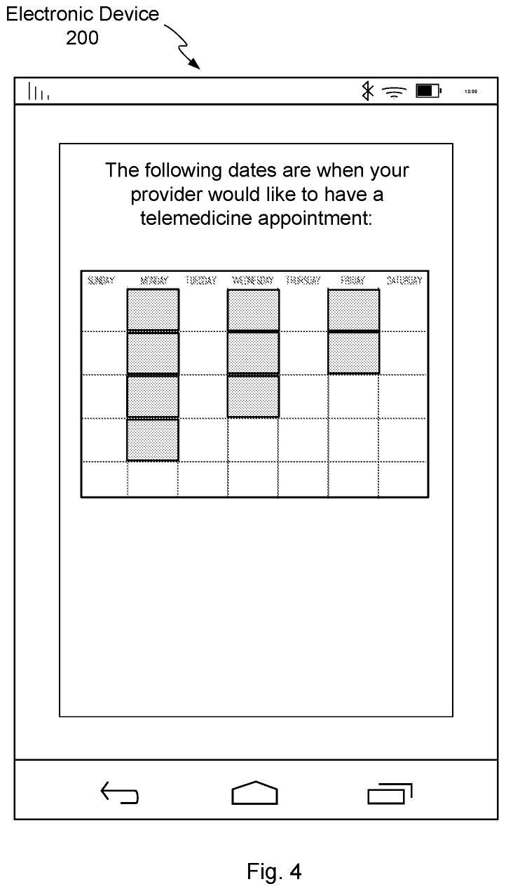

FIG. 4 shows an exemplary screenshot of a user interface presented to a patient including dates that a provider would like to meet with the patient. For example, after a procedure (e.g., rhinoplasty), a provider may want to meet with a patient three times the week after the procedure, three times the following week, twice the week after that, and then once the week after that. In some embodiments, health care platform 100 (FIG. 1) may suggest the dates based on the type of procedure/operation performed, the provider's schedule, the patient's schedule, etc.

In some embodiments, a patient or a provider may be able to reschedule appointments, appointment times, or otherwise change the time of appointments prior to, or following a procedure/operation/surgery.

In some embodiments, the suggested dates presented to a user may be based on an input from a third-party scheduling tool (e.g., Calendly™, Google Calendar™, Microsoft Office™). In such a case, information may be received from a user or provider's third-party scheduling tool's account. Such information may be collected by a user or provider's electronic device (e.g., their smartphone or personal computer) and then provided to a server running at least a portion of the healthcare platform 100. In some embodiments, the suggested dates presented to a user may be based on a time of day, a location of a user or a user's device, the size of a wound, the type of wound, the medications that the user has been prescribed, a user's medical history, a provider's availability, and/or a condition of a wound.

In one or more embodiments, notifications may be sent to caretakers (family, friends, hired individuals/services) of the patient that provide updates. For example, notifications may be sent based upon patient dropoff reminder time, procedure start, mid procedure updates, procedure end, and/or patient release time. The patient likely will have had to provide approval/consent for such notifications to be sent.

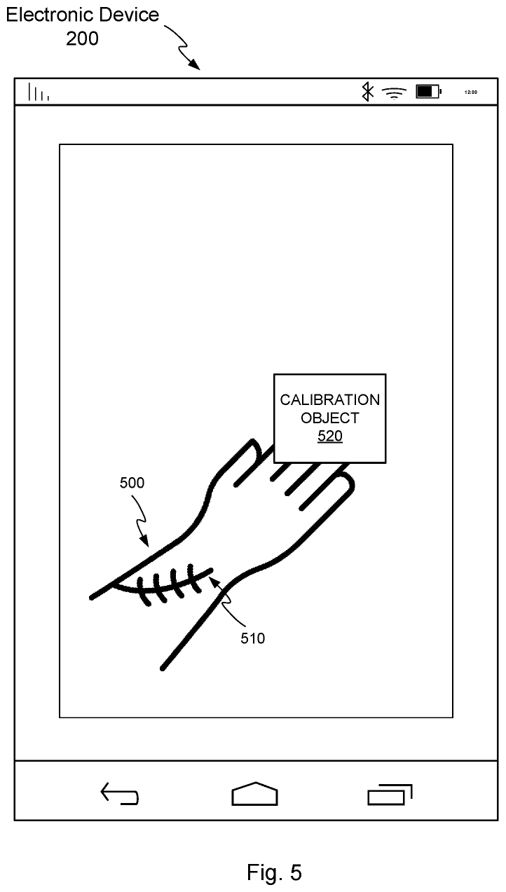

FIG. 5 shows an exemplary image taken by a patient of a body part on an electronic device 200. The image shows a patient's arm 500, a wound 510, and a calibration object 520. Such an image may allow a provider to see whether wound 510 is properly healing during a telemedicine appointment following a procedure.

In some embodiments, calibration object 520 (which may be a white card or other color/pattern, an object of a certain size, a fiducial, etc.) may be used at least in part to cause an image to be shown with the correct size and/or lighting. For example, since cameras and screens can vary so drastically in their brightness—which may cause a provider or system to incorrectly determine whether a wound abnormality exists—an object of a known brightness may be used to calibrate the image such that any brightness, contrast, shadow correction, or other adjustments may be made to avoid an incorrect determination. Further, if the size of the calibration object 520 is known by healthcare platform 100 (FIG. 1), then the healthcare platform may determine the size of the wound 510 to determine any abnormalities.

In some embodiments, a user interface may provide instructions to a user including how to capture an image of the wound 510. For example, a user interface may provide information about the calibration object 520. For example, the user interface may show information indicating what size the calibration object should be (e.g., 3 inch×3 inch, or 5 inch×5 inch). As another example, a user interface may provide instructions on whether the lighting is correct, including whether the image is too dark. As another example, the health platform 100 may identify the type of wound, and the user interface may instruct a user to provide certain information about the wound, including written information and an image. For example, the health platform 100 may identify the type of wound, and provide instructions indicating what part of the wound a user should take a picture of, an angle that the user should take a picture of, etc.

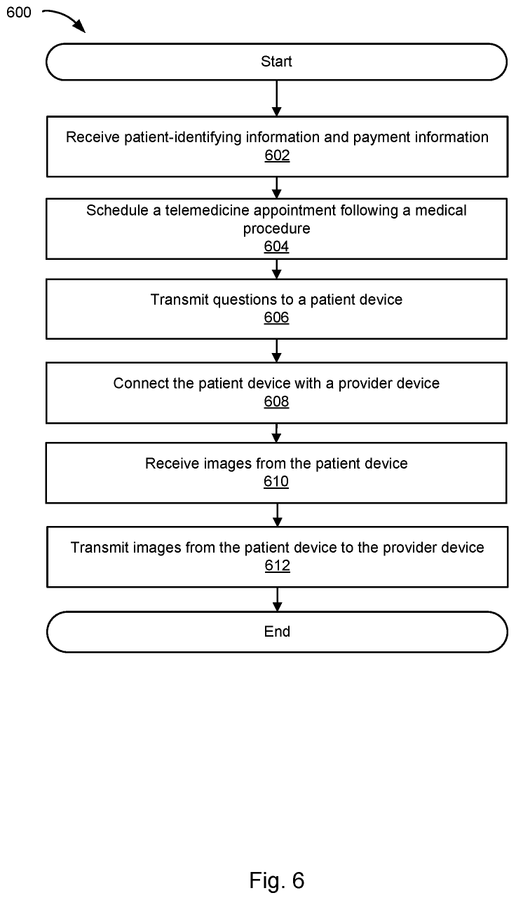

FIG. 6 shows a flowchart 600 of a method for scheduling an appointment. While the various steps in this flowchart are presented and described sequentially, one of ordinary skill will appreciate that some or all of the steps can be executed in different orders and some or all of the steps can be executed in parallel. Further, in one or more embodiments, one or more of the steps described below can be omitted, repeated, and/or performed in a different order. Accordingly, the specific arrangement of steps shown in FIG. 6 should not be construed as limiting the scope of the invention.

At STEP 602, patient-identifying information and payment information is received. For example, payment from a patient may be made online, or via a 3^{rd} party provider. In some embodiments, an insurance carrier may approve the payment.

At STEP 604, a telemedicine appointment is scheduled for after a medical procedure. Herein, the terms procedure, operation, and surgery may be used interchangeably, as would be known by a person of ordinary skill in the art. The appointment may also, in some cases, be scheduled before a medical procedure. In various embodiments, a telemedicine appointment scheduled for after a procedure may be used for the provider to ask the patient questions, see video or images of a wound, or otherwise determine the wellbeing of a patient.

At STEP 606, questions are transmitted to a patient device. Such questions may be used to modify electronic medical records. In such an embodiment, the modifications to their EMR may be made such that a medical assistant doesn't need to waste time asking them, and the patient may update their EMR to prevent EMR data from becoming stale (i.e., too old as to become incorrect).

At STEP 608, a patient device is connected with a provider device. The healthcare platform 100 (FIG. 1) may facilitate this connection, and allow a patient to securely discuss the status of their recovery with their provider. Using telemedicine, a patient does not have to commute across town for an appointment which may be performed online/via telemedicine.

At STEP 610, images are received from a patient device. For example, healthcare platform 100 (FIG. 1), a provider's device, and/or an administrator's device may receive images that show a wound or an area of a patient's body prior to a medical procedure. In some embodiments, AI and/or machine learning may be used to assist in determining whether a wound is healing correctly by comparing the images sent by a patient to a database or other application that is applied by healthcare platform 100 (FIG. 1).

At STEP 612, images from the patient device are sent to the provider device. As described above, the images may be received by the provider, who may then determine whether a wound or other injury is healing correctly (e.g., a dislocation).

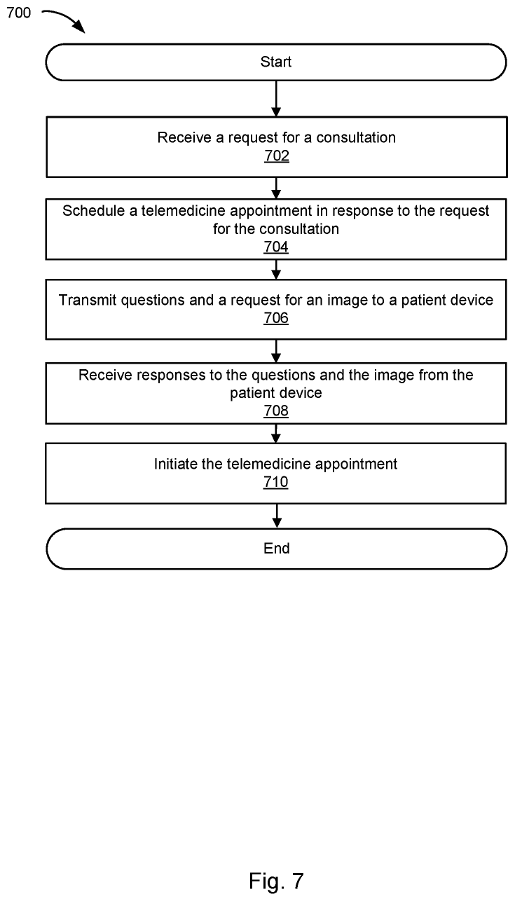

FIG. 7 shows a flowchart 700 of a method for scheduling an appointment. While the various steps in this flowchart are presented and described sequentially, one of ordinary skill will appreciate that some or all of the steps can be executed in different orders and some or all of the steps can be executed in parallel.

For example, all of the steps in flowchart 700 may occur after a medical procedure takes place. In such an embodiment, the request for a consultation/appointment could instead be considered a request for a follow-up telemedicine visit.

Further, in one or more embodiments, one or more of the steps described below can be omitted, repeated, and/or performed in a different order. Accordingly, the specific arrangement of steps shown in FIG. 7 should not be construed as limiting the scope of the invention.

At STEP 702, a request for a consultation may be received. For example, healthcare platform 100 may receive a request for an appointment from a patient device, or a device belonging to a family member or caretaker of the patient. In some embodiments, the request may come from another provider (e.g., similar to a referral).

At STEP 704, a telemedicine appointment is scheduled in response to the request for the consultation. In some embodiments, the appointment may be scheduled automatically, without human intervention. Whether an appointment is scheduled automatically may depend on the type of consultation requested (e.g., the severity of the medical condition). In some embodiments, the appointment may be scheduled by a provider or a patient.

At STEP 706, questions and a request for an image are sent to a patient. Such questions may include a patient's medical history, a request for information associated with a patient's EMR, a patient's identifying information, a patient's insurance information, and a patient's contact information. In some embodiments, a request for an image is also presented to a patient. This request may specify an area of the body that the image should be taken of. For example, healthcare platform 100 may request a picture of the face of a patient. The area of the body of which an image is requested may be based on the type of consultation requested.

At STEP 708, one or more responses to the questions and/or an image are provided from the patient device. These responses may be sent to a server and/or a provider's device. A telemedicine appointment may be scheduled based on the responses to the questions and/or the image. For example, if the responses indicate that the patient is in severe pain or at risk of further injury, the telemedicine appointment may be scheduled for an earlier time than it would be for a less severe condition.

At STEP 710, the telemedicine appointment is initiated. In various embodiments, the telemedicine appointment will not be initiated if the responses/answers to the questions and/or one or more images are not provided. For example, even though a consultation/appointment may be scheduled, it may not begin unless the requested answers to questions and/or image are provided by a patient.

Thus, as in various embodiments described herein, the instant application may describe a method, comprising receiving, at a server, patient-identifying information and payment information. For example, a server that comprises at least a portion of healthcare platform 100 may receive patient-identifying information and payment information. Such patient identifying information may include a social security number, a birthday, or a phone number. Payment information may include credit card information, or bank information such as a routing number and an account number.

Further, in some embodiments, the method may further comprise scheduling, at the server, a telemedicine appointment following a medical procedure. For example, the healthcare platform 100, which at least a portion of may be running on the server, may schedule one or more telemedicine appointments in the healthcare platform, and/or add those appointments to a user and/or provider's third-party calendaring software. Although the healthcare platform 100 may schedule a telemedicine appointment following the medical procedure, it should be understood that healthcare platform 100 can schedule a telemedicine appointment prior to the medical procedure.

Further, in some embodiments, the method may further comprise transmitting questions to a patient device, wherein the questions are associated with the medical procedure. For example, before and/or after a medical procedure, a user's device may receive information from the server, which each may be running at least a portion of the healthcare platform 100, which may include, wherein the information includes questions or other prompts. These may instruct a user to enter how much pain they have that day, what they've eaten recently, how they are feeling on a scale of 1-10, what color a wound is, how many medicines they've taken that day or over a period of time, etc.

Further, in some embodiments, the method may further comprise, connecting the patient device with a provider device in response to receiving answers to the questions from the patient device. For example, a patient device may be a smart phone or computer, a provider device may be a smartphone or computer, and a server may be separate from those two devices (and in some cases part of a cloud). The patient device may then be communicatively connected/coupled to the provider device in response to receiving answers to the questions described above. The connection between the patient device and the provider device may occur shortly after the patient answers the questions, or may occur any time after the patient answers the questions (e.g., a day or week later).

Further, in some embodiments, the method may further comprise receiving images from the patient device. For example, a patient may provide images to a server and/or a provider's device. These images may be of wounds. Herein, wounds may be used to describe infections, lacerations, abrasions, blunt trauma, and/or punctures. In some embodiments, the images received from the patient device may include images without wounds. For example, a patient may have a wound in one area of their body, and the patient may submit (or be requested to submit) an image of another area of their body (e.g., this may be done so a provider may compare the images such as a picture of one arm with a wound and another arm without the wound).

Further, in some embodiments, the method may further comprise transmitting the images from the patient device to the provider device. For example, the images may be sent from a patient device to healthcare platform 100 (if the patient device is not considered part of healthcare platform 100), and then the image may be sent from healthcare platform 100 to the provider device (if the provider device is not considered part of healthcare platform 100). In other words, a patient may send an image from their device, to a server, and the server may send the image to a provider's device. In addition or alternatively, an image may be sent from a patient device to a provider device without going through a server.

Further, in some embodiments, the method may further comprise scheduling, at the server, a telemedicine appointment prior to the medical procedure. The telemedicine appointment may be scheduled for a time prior to or after a medical procedure. In some cases, the appointment may be scheduled at a time chosen at the patient device, or it may be scheduled at a time chosen at the provider device. In some embodiments, healthcare platform 100 may suggest appointment times and/or schedule them based on information such as the type of procedure to be/that was performed, an amount of time after and/or before the procedure (e.g., 2 days after the procedure), a characteristic of the patient (e.g., if the patient is pregnant or has an immunodeficiency condition), and/or information retrieved from third-party software such as Google Calendar™. In some embodiments, the embodiment may change based on information entered in response to questions at a patient device, and/or in response to information entered into a patient's EMR by a provider. For example, if a patient's wound is not healing as well as desired, a provider may enter such information into a patient's EMR, and health platform 100 may schedule and/or change the time of a telemedicine appointment (e.g., make it a week earlier) based at least in part on the information entered by the provider.

Further, in some embodiments, the method may further comprise determining an abnormality associated with a patient recovery based at least in part on the responses to the questions. Such a determination may occur at a server, where at least part of healthcare platform 100 is located. Such an abnormality may be determined by an algorithm that implements machine learning or one or more neural networks. Questions that are presented to a user may include how much pain the user is experiencing, whether the wound is secreting anything such as pus, whether the wound and/or an area surrounding the wound is raised, and/or whether the wound is discolored (e.g., red, blue, black, or some combination thereof).

Further, in some embodiments, the method may further comprise determining an abnormality associated with a patient recovery based at least in part on an image of a wound. Such a determination may occur at a server, where at least part of healthcare platform 100 is located. As described above, healthcare platform 100 may determine an abnormality, such as an infection or undesired healing based on an image taken of a wound.

In some embodiments, the abnormality may be determined based at least in part upon a comparison of the image of the wound and other images of other wounds using a machine learning algorithm. In other words, an algorithm may determine one or more abnormalities, wherein the algorithm was created at least in part using machine learning. For example, a machine learning model may be created using images (e.g., of wounds) and the resulting algorithm may find patterns in the images that are then used to compare to the image of the wound.

In some embodiments, the responses to the questions from the patient device are inputted into an electronic medical record belonging to the patient. For example, one or more electronic medical records may be associated with a patient, information associated with an image of a wound, a provider's notes or recommendations about the wound, and/or information about a telemedicine appointment may be inputted into the electronic medical records. These electronic medical records may also contain information associated with a patient's medical history, what medications the patient is currently taking, and responses to questions provided by the patient that are associated with a patient's wound.

Further, in some embodiments, the method may further comprise providing information about the patient associated with their recovery. This information may be provided to a family member device (e.g., a device other than the patient's device, the server, or the provider's device). For example, healthcare platform 100 and/or a provider device may provide information about the patient to a device other than the patient's device. Such a family member device may be associated with a parent, a sibling, a child, or other relative of a patient. In some embodiments the family member device may submit information associated with a patient's recovery to a non-family member, such as a caregiver or insurance company. It should be understood that information about a patient other than their recovery (e.g., what time a medical procedure will occur, when a patient's telemedicine appointments are scheduled for, a type of medicine a patient should take) may be provided to a family member's device. Such information may be provided prior to the patient's medical procedure.

Further, in some embodiments, the method may further comprise scheduling, at the server, a plurality of telemedicine appointments following the medical procedure. Rather than simply schedule a single telemedicine appointment, telemedicine platform (e.g., healthcare platform 100) may schedule multiple appointments. The way these appointments are scheduled may be based on a variety of factors discussed herein, and the time these appointments are scheduled may be based on a variety of factors discussed herein.

For example, the time of the plurality of telemedicine appointments may be based in part on the type of medical procedure. If a procedure is to remove a wart, for example, only one telemedicine appointment may be scheduled. In another embodiment, if a procedure is for a fractured arm, multiple appointments may be scheduled. In some embodiments such scheduling may be automatic by the system, and/or manually scheduled by a person.

In some embodiments, the time of the scheduled telemedicine appointments is based in part on a pain level of the patient. For example, if a patient is experiencing extreme pain on a certain day after a procedure, a telemedicine appointment may be moved to an earlier time. Similarly, if a patient is experiencing a certain amount of pain prior to a procedure, a telemedicine appointment, or the procedure itself, may have its time changed by health platform 100. In some embodiments, at least part of the changing of the time of an appointment may be performed at a client device or a provider's device.

In some embodiments, the images include wounds that are healing from the medical procedure. As described above, the images may include wounds that are healing from a medical procedure, and be used by a provider to diagnose potential issues such as an infection or other abnormality.

Embodiments described herein may be discussed in the general context of computer-executable instructions residing on some form of computer-readable storage medium, such as program modules, executed by one or more computers or other devices. By way of example, and not limitation, computer-readable storage media may comprise non-transitory computer-readable storage media and communication media; non-transitory computer-readable media include all computer-readable media except for a transitory, propagating signal. Generally, program modules include routines, programs, objects, components, data structures, etc., that perform particular tasks or implement particular abstract data types. The functionality of the program modules may be combined or distributed as desired in various embodiments.

Computer storage media includes volatile and nonvolatile, removable and non-removable media implemented in any method or technology for storage of information such as computer-readable instructions, data structures, program modules or other data. Computer storage media includes, but is not limited to, random access memory (RAM), read only memory (ROM), electrically erasable programmable ROM (EEPROM), flash memory or other memory technology, compact disk ROM (CD-ROM), digital versatile disks (DVDs) or other optical storage, magnetic cassettes, magnetic tape, magnetic disk storage or other magnetic storage devices, or any other medium that can be used to store the desired information and that can be accessed to retrieve that information.

Communication media can embody computer-executable instructions, data structures, and program modules, and includes any information delivery media. By way of example, and not limitation, communication media includes wired media such as a wired network or direct-wired connection, and wireless media such as acoustic, radio frequency (RF), infrared, and other wireless media. Combinations of any of the above can also be included within the scope of computer-readable media.

Embodiments may be implemented on a specialized computer system. The specialized computing system can include one or more modified mobile devices (e.g., laptop computer, smart phone, personal digital assistant, tablet computer, or other mobile device), desktop computers, servers, blades in a server chassis, or any other type of computing device(s) that include at least the minimum processing power, memory, and input and output device(s) to perform one or more embodiments.

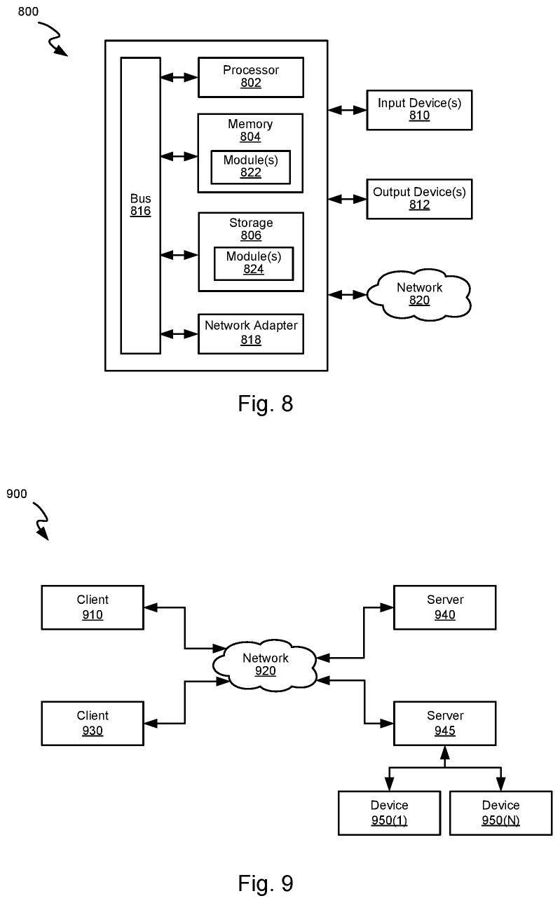

For example, as shown in FIG. 8, the computing system 800 may include one or more computer processor(s) 802, associated memory 804 (e.g., random access memory (RAM), cache memory, flash memory, etc.), one or more storage device(s) 806 (e.g., a hard disk, an optical drive such as a compact disk (CD) drive or digital versatile disk (DVD) drive, a flash memory stick, etc.), a bus 816, and numerous other elements and functionalities. The computer processor(s) 802 may be an integrated circuit for processing instructions. For example, the computer processor(s) may be one or more cores or micro-cores of a processor.

In one or more embodiments, the computer processor(s) 802 may be an integrated circuit for processing instructions. For example, the computer processor(s) 802 may be one or more cores or micro-cores of a processor. The computer processor(s) 802 can implement/execute software modules stored by computing system 800, such as module(s) 822 stored in memory 804 or module(s) 824 stored in storage 806. For example, one or more of the modules described herein can be stored in memory 804 or storage 806, where they can be accessed and processed by the computer processor 802. In one or more embodiments, the computer processor(s) 802 can be a special-purpose processor where software instructions are incorporated into the actual processor design.

The computing system 800 may also include one or more input device(s) 810, such as a touchscreen, keyboard, mouse, microphone, touchpad, electronic pen, or any other type of input device. Further, the computing system 800 may include one or more output device(s) 812, such as a screen (e.g., a liquid crystal display (LCD), a plasma display, touchscreen, or other display device), a printer, external storage, or any other output device. The computing system 800 may be connected to a network 820 (e.g., a local area network (LAN), a wide area network (WAN) such as the Internet, mobile network, or any other type of network) via a network interface connection 818. The input and output device(s) may be locally or remotely connected (e.g., via the network 820) to the computer processor(s) 802, memory 804, and storage device(s) 806.

One or more elements of the aforementioned computing system 800 may be located at a remote location and connected to the other elements over a network 820. Further, embodiments may be implemented on a distributed system having a plurality of nodes, where each portion may be located on a subset of nodes within the distributed system. In one embodiment, the node corresponds to a distinct computing device. Alternatively, the node may correspond to a computer processor with associated physical memory. The node may alternatively correspond to a computer processor or micro-core of a computer processor with shared memory and/or resources.

For example, one or more of the software modules disclosed herein may be implemented in a cloud computing environment. Cloud computing environments may provide various services and applications via the Internet. These cloud-based services (e.g., software as a service, platform as a service, infrastructure as a service, etc.) may be accessible through a Web browser or other remote interface.

One or more elements of the above-described systems may also be implemented using software modules that perform certain tasks. These software modules may include script, batch, routines, programs, objects, components, data structures, or other executable files that may be stored on a computer-readable storage medium or in a computing system. These software modules may configure a computing system to perform one or more of the example embodiments disclosed herein. The functionality of the software modules may be combined or distributed as desired in various embodiments. The computer readable program code can be stored, temporarily or permanently, on one or more non-transitory computer readable storage media. The non-transitory computer readable storage media are executable by one or more computer processors to perform the functionality of one or more components of the above-described systems and/or flowcharts. Examples of non-transitory computer-readable media can include, but are not limited to, compact discs (CDs), flash memory, solid state drives, random access memory (RAM), read only memory (ROM), electrically erasable programmable ROM (EEPROM), digital versatile disks (DVDs) or other optical storage, and any other computer-readable media excluding transitory, propagating signals.

FIG. 9 is a block diagram of an example of a network architecture 900 in which client systems 910 and 930, and servers 940 and 945, may be coupled to a network 920. Network 920 may be the same as or similar to network 920. Client systems 910 and 930 generally represent any type or form of computing device or system, such as client devices (e.g., portable computers, smart phones, tablets, smart TVs, etc.).

Similarly, servers 940 and 945 generally represent computing devices or systems, such as application servers or database servers, configured to provide various database services and/or run certain software applications. Network 920 generally represents any telecommunication or computer network including, for example, an intranet, a wide area network (WAN), a local area network (LAN), a personal area network (PAN), or the Internet.

With reference to computing system 800 of FIG. 8, a communication interface, such as network adapter 818, may be used to provide connectivity between each client system 910 and 930, and network 920. Client systems 910 and 930 may be able to access information on server 940 or 945 using, for example, a Web browser, thin client application, or other client software. Such software may allow client systems 910 and 930 to access data hosted by server 940, server 945, or storage devices 950(1)-(N). Although FIG. 9 depicts the use of a network (such as the Internet) for exchanging data, the embodiments described herein are not limited to the Internet or any particular network-based environment.

In one embodiment, all or a portion of one or more of the example embodiments disclosed herein are encoded as a computer program and loaded onto and executed by server 940, server 945, storage devices 950(1)-(N), or any combination thereof. All or a portion of one or more of the example embodiments disclosed herein may also be encoded as a computer program, stored in server 940, run by server 945, and distributed to client systems 910 and 930 over network 920.

Although components of one or more systems disclosed herein may be depicted as being directly communicatively coupled to one another, this is not necessarily the case. For example, one or more of the components may be communicatively coupled via a distributed computing system, a cloud computing system, or a networked computer system communicating via the Internet.

And although only one computer system may be depicted herein, it should be appreciated that this one computer system may represent many computer systems, arranged in a central or distributed fashion. For example, such computer systems may be organized as a central cloud and/or may be distributed geographically or logically to edges of a system such as a content/data delivery network or other arrangement. It is understood that virtually any number of intermediary networking devices, such as switches, routers, servers, etc., may be used to facilitate communication.

While the invention has been described with respect to a limited number of embodiments, those skilled in the art, having benefit of this disclosure, will appreciate that other embodiments may be devised that do not depart from the scope of the invention as disclosed herein.

While the present disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered as examples because other architectures can be implemented to achieve the same functionality.

The process parameters and sequence of steps described and/or illustrated herein are given by way of example only. For example, while the steps illustrated and/or described herein may be shown or discussed in a particular order, these steps do not necessarily need to be performed in the order illustrated or discussed. Some of the steps may be performed simultaneously. For example, in certain circumstances, multitasking and parallel processing may be advantageous. The various example methods described and/or illustrated herein may also omit one or more of the steps described or illustrated herein or include additional steps in addition to those disclosed.

It is understood that a “set” can include one or more elements. It is also understood that a “subset” of the set may be a set of which all the elements are contained in the set. In other words, the subset can include fewer elements than the set or all the elements of the set (i.e., the subset can be the same as the set).

What is claimed is:

1. A method, comprising:
receiving, at a server, patient-identifying information and payment information;
scheduling, at the server, a telemedicine appointment following a medical procedure;
transmitting questions to a patient device, wherein the questions are associated with the medical procedure;
in response to receiving answers to the questions from the patient device, connecting the patient device with a provider device;
receiving images from the patient device; and
transmitting the images from the patient device to the provider device.

2. The method of claim 1, further comprising:
scheduling, at the server, a telemedicine appointment prior to the medical procedure.

3. The method of claim 1, further comprising:
determine, at the server, an abnormality associated with a patient recovery based at least in part on the responses to the questions.

4. The method of claim 1, further comprising:
determining, at the server, an abnormality associated with a patient recovery based at least in part on an image of a wound.

5. The method of claim 5, wherein determining the abnormality is based at least in part upon a comparison of the image of the wound and other images of other wounds using a machine learning algorithm.

6. The method of claim 1, wherein the responses to the questions from the patient device are inputted into an electronic medical record belonging to the patient.

7. The method of claim 1, further comprising:
providing, to a family member device, information about the patient associated with their recovery.

8. The method of claim 1, further comprising:
scheduling, at the server, a plurality of telemedicine appointments following the medical procedure.

9. The method of claim 8, wherein time of the plurality of telemedicine appointments is based in part on the medical procedure.

10. The method of claim 8, wherein the time of the plurality of telemedicine appointments is based in part on a pain level of the patient.

11. The method of claim 1, wherein the images include wounds that are healing from the medical procedure.

12. A system for scheduling treatments, the system comprising:
a computer processor;
a memory; and
a telemedicine scheduling engine executing on the computer processor and configured to:
receive, at a first electronic device, patient-identifying information and payment information;
schedule, at the first electronic device, a telemedicine appointment following a medical procedure;
transmit, from the first electronic device, questions to a patient device, wherein the questions are associated with the medical procedure;
in response to receiving answers to the questions from the patient device, cause to connect, at the first electronic device, the patient device with a provider device;
receive, at the first electronic device, images from the patient device; and
transmit, from the first electronic device, images from the patient device to the provider device.

13. The system of claim 12, wherein the telemedicine scheduling engine executing on the computer processor is further configured to:
schedule, at the server, a telemedicine appointment prior to the medical procedure.

14. The system of claim 12, wherein the telemedicine scheduling engine executing on the computer processor is further configured to:
determine, at the server, an abnormality associated with a patient recovery based at least in part on the responses to the questions.

15. The system of claim 12, wherein the telemedicine scheduling engine executing on the computer processor is further configured to:
determine, at the server, an abnormality associated with a patient recovery based at least in part on an image of a wound.

16. The system of claim 15, wherein determining the abnormality is based at least in part upon a comparison of the wound and other images of other wounds using a machine learning algorithm.

17. The system of claim 12, wherein the responses to the questions from the patient device are inputted into an electronic medical record belonging to the patient.

18. The system of claim 12, wherein the telemedicine scheduling engine executing on the computer processor is further configured to:
provide, to a family member device, information about the patient associated with their recovery.

19. The system of claim 12, wherein the telemedicine scheduling engine executing on the computer processor is further configured to:
schedule, at the server, a plurality of telemedicine appointments following the medical procedure.

20. The system of claim 19, wherein the time of the plurality of telemedicine appointments is based in part on the medical procedure, and wherein the time of the plurality of telemedicine appointments is based in part on a pain level of the patient.
